(19) United States
(12) Patent Application Publication
(10) Pub. No.: US 2025/0009447 A1
(43) Pub. Date: Jan. 9, 2025

(54) DOUBLE DELTA STRUCTURE OF SURGICAL ROBOT

(71) Applicant: ROEN Surgical, Inc., Daejeon (KR)

(72) Inventors: Un-Je YANG, Daejeon (KR); Chang-Kyun KIM, Daejeon (KR); Duk-Yoo KONG, Daejeon (KR); Duk-Sang KIM, Daejeon (KR)

(73) Assignee: ROEN Surgical, Inc., Daejeon (KR)

(21) Appl. No.: 18/709,680

(22) PCT Filed: Nov. 14, 2022
(86) PCT No.: PCT/KR2022/017870

§ 371 Date: Aug. 1, 2024

(30) Foreign Application Priority Data
Nov. 16, 2021 (KR) 10-2021-0157988

(51) Int. Cl. A61B 34/30; A61B 34/00; A61B 90/50

(52) U.S. Cl. CPC A61B 34/30; A61B 34/70; A61B 2090/506

(57) ABSTRACT

The present invention relates to a double delta structure of a surgical robot, and to a double delta structure of a surgical robot, which provides a link coupling structure for the double delta structure so as to be resistant against distortion or external force. To this end, disclosed is the double delta structure of a surgical robot, the structure comprising: a first link part link-coupled so as to pitch and yaw around a reference axis in a first direction; a second link part link-coupled so as to pitch and yaw around a reference axis in a second direction; and a third link part link-coupled so as to pitch and yaw around a reference axis in a third direction.

### TECHNICAL FIELD

The present disclosure relates to a double delta structure of a surgical robot and, more particularly, to a double delta structure of a surgical robot which provides a link coupling structure for a double delta structure to be resistant to torsion or external force.

### BACKGROUND ART

A general three-degree-of-freedom link parallel structure has a low degree of freedom due to its high rigidity but low workspace (or a work area), but a delta structure, which can have a high workspace, is being widely adopted.

However, the conventional delta structure is a structure widely used in industrial sites and is generally arranged horizontally to a floor to be used, and thus is not required to consider the effect of moments.

On the other hand, since a surgical robot is required to be placed at various inclinations depending on a surgical site, there is a problem in that desired precision and sufficient rigidity cannot be secured when using the conventional delta structure.

### PRIOR ART DOCUMENT

(Patent Document 0001) KR 10-2018-0001153

### DISCLOSURE

### Technical Problem

Accordingly, the present disclosure has been devised to solve the above-mentioned problems and is intended to provide a structure that is resistant to torsional moments by doubly coupling a delta structure.

However, the purposes of the present disclosure are not limited to the purposes mentioned above, and other purposes not mentioned will be clearly understood by those skilled in the art from the description below.

### Technical Solution

The objectives of the present disclosure described above are achieved by providing a double delta structure of a surgical robot, the structure including a first link part link-coupled to perform pitch and yaw rotations around a reference axis in a first direction, a second link part link-coupled to perform pitch and yaw rotations around a reference axis in a second direction, and a third link part link-coupled to perform pitch and yaw rotations around a reference axis in a third direction.

In addition, a first delta structure may be formed through link connections of upper sides of the first, second, and third link parts, and a second delta structure may be formed through link connections of lower sides of the first, second, and third link parts so as to form a double delta structure.

In addition, each of the first, second, and third link parts may be composed of identical pairs of links and may be link-coupled to form a parallelogram shape.

In addition, each of the first, second, and third link parts may be link-coupled such that a plurality of parallelogram shapes are parallel to each other.

In addition, first, second, and third parallelograms formed on each of the first, second, and third link parts may be formed to face each other while being spaced apart from each other at predetermined angles.

In addition, each of the first, second, and third link parts may include a plurality of base link parts, a plurality of upper joint parts link-coupled to rotate on upper sides of the base link parts, a plurality of upper restraining link parts link-coupled to the upper joint parts to perform pitch and yaw rotations, a plurality of lower joint parts link-coupled to rotate on lower sides of the base link parts, and a plurality of lower restraining link parts link-coupled to the lower joint parts to perform pitch and yaw rotations.

In addition, first and second upper restraining link parts may be link-coupled parallel to each other to the plurality of upper joint parts to perform pitch and yaw rotations, and first and second lower restraining link parts may be link-coupled parallel to each other to the plurality of lower joint parts to perform pitch and yaw rotations.

In addition, the structure may further include a first parallelogram formed by comprising the first upper restraining link part and the first lower restraining link part, and a second parallelogram formed by comprising the second upper restraining link part and the second lower restraining link part.

In addition, the first parallelogram and the second parallelogram may be formed parallel to each other.

In addition, the plurality of base link parts may include a first base link part to which a first upper joint part and a first lower joint part are link-coupled to be rotatable, and a second base link part to which a second upper joint part and a second lower joint part are link-coupled to be rotatable.

In addition, opposite-side coupling links of first and second upper restraining link parts may be link-coupled to the first and second upper joint parts, respectively, and opposite-side coupling links of first and second lower restraining link parts may be link-coupled to the first and second lower joint parts, respectively.

In addition, the second base link part may further include a moving shaft coupling part that moves along a moving shaft, and a moving frame formed by combination of the first, second, and third link parts may be capable of performing three degrees of freedom movement by moving along the moving shaft.

### Advantageous Effects

According to the present disclosure as described above, there is an effect of providing a structure that is resistant to torsional moments by doubly coupling a delta structure.

### DESCRIPTION OF DRAWINGS

The following drawings attached to the present disclosure illustrate a preferred embodiment of the present disclosure and, along with the detailed description of the present disclosure, serve to further help the understanding of the technical spirit of the present disclosure. Therefore, the present disclosure should not be construed as being limited to the details set forth in such drawings.

FIGS. 1 and 2 are views schematically illustrating a double delta structure of a surgical robot according to an embodiment of the present disclosure,

FIG. 3 is a view illustrating a first link part according to an embodiment of the present disclosure,

FIG. 4 is a view illustrating a pitch rotation (θ₁) and a yaw rotation (θ₂) performed by first and second upper restraining link parts and first and second lower restraining link parts of the first link part according to an embodiment of the present disclosure, compared to FIG. 3,

FIG. 5 is a view illustrating that each of second base link parts of first, second, and third link parts 100, 200, and 300 according to an embodiment of the present disclosure moves up and down along a moving shaft coupled thereto.

### BEST MODE

The objective of the present disclosure may be achieved by providing a double delta structure of a surgical robot, the structure including: a first link part link-coupled to perform pitch and yaw rotations around a reference axis in a first direction, a second link part link-coupled to perform pitch and yaw rotations around a reference axis in a second direction, and a third link part link-coupled to perform pitch and yaw rotations around a reference axis in a third direction.

### MODE FOR INVENTION

Hereinafter, a preferred embodiment of the present disclosure will be described with reference to the drawings. In addition, an embodiment described below does not unduly limit the content of the present disclosure described in the patent claims, and it cannot be said that the entire configuration described in this embodiment is essential as a means of resolving the present disclosure. In addition, description of prior arts and matters that are obvious to those skilled in the art may be omitted, and the omitted descriptions of components (methods) and functions may be sufficiently referenced within the scope of the technical idea of the present disclosure.

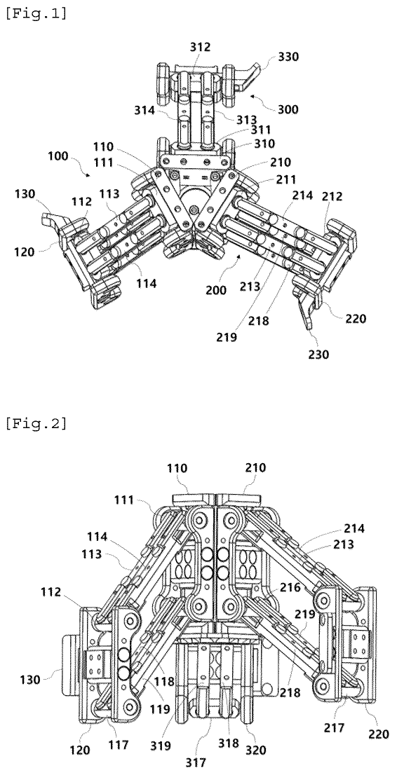

A double delta structure of a surgical robot (or a parallel delta structure of a surgical robot) according to an embodiment of the present disclosure includes first, second, and third link parts 100, 200, and 300 as illustrated in FIGS. 1 and 2. Since the first, second, and third link parts 100, 200, and 300 are made of identical link members, only a first link part 100 will be described below, and the description of the second and third link parts 200 and 300 will be replaced with the description of the first link part 100.

As illustrated in FIGS. 1 and 2, the first, second, and third link parts 100, 200, and 300 according to an embodiment of the present disclosure are each oriented at approximately 120 degrees. As an example, the first link part 100 is disposed in a first direction, a second link part 200 is disposed in a second direction 120 degrees away from the first direction, and a third link part 300 is disposed in a third direction 120 degrees away from the second direction.

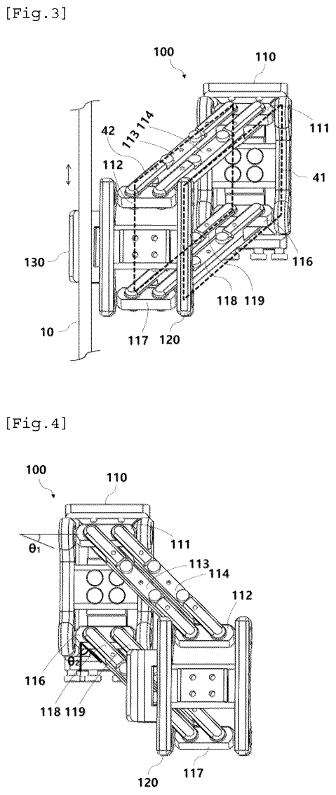

As illustrated in FIGS. 3 and 4, the first link part 100 according to an embodiment of the present disclosure includes
a first base link part 110 and a second base link part 120.

The first base link part 110 of the first link part 100, a first base link part 210 of the second link part 200, and a first base link part 310 of the third link part 300 are coupled to each other on the center of a moving frame to form an approximately triangular shape as illustrated in FIG. 1.

The first base link part 110 and the second base link part 120 are arranged to face each other at a predetermined distance apart.

A first upper joint part 111 is rotatably link-coupled to the upper side of the first base link part 110. A first lower joint part 116 is rotatably link-coupled to the lower side of the first base link part 110. A second upper joint part 112 is rotatably link-coupled to the upper side of the second base link part 120. A second lower joint part 117 is rotatably link-coupled to the lower side of the second base link part 120.

A first upper restraining link part 113 includes a first restraining joint part provided on a first end part thereof and includes a second restraining joint part provided on a second end part thereof. The first restraining joint part of the first upper restraining link part 113 is restrainedly coupled to the first upper joint part 111, and the second restraining joint part thereof is restrainedly coupled to the second upper joint part 112.

Meanwhile, a second upper restraining link part 114 includes a first restraining joint part provided on a first end part thereof and includes a second restraining joint part provided on a second end part thereof. The first restraining joint part of the second upper restraining link part 114 is restrainedly coupled to the first upper joint part 111, and the second restraining joint part thereof is restrainedly coupled to the second upper joint part 112. That is, each of the first and second upper restraining link parts 113 and 114 is restrainedly coupled parallel to each other to the first and second upper joint parts 111 and 112.

A first lower restraining link part 118 includes a first restraining joint part provided on a first end part thereof and includes a second restraining joint part provided on a second end part thereof. The first restraining joint part of the first lower restraining link part 118 is restrainedly coupled to the first lower joint part 116, and the second restraining joint part thereof is restrainedly coupled to the second lower joint part 117.

Meanwhile, a second lower restraining link part 119 includes a first restraining joint part provided on a first end part thereof and includes a second restraining joint part provided on a second end part thereof. The first restraining joint part of the second lower restraining link part 119 is restrainedly coupled to the first lower joint part 116, and the second restraining joint part thereof is restrainedly coupled to the second lower joint part 117. That is, each of the first and second lower restraining link parts 118 and 119 is restrainedly coupled parallel to each other to the first and second lower joint parts 116 and 117.

In this case, the first and second upper joint parts 111 and 112 and the first and second lower joint parts 116 and 117 may each rotate in the first direction relative to the first and second base link parts 110 and 120. In addition, the first and second upper restraining link parts 113 and 114 and the first and second lower restraining link parts 118 and 119 may each rotate in the second direction relative to the first and second upper joint parts 111 and 112 and the first and second lower joint parts 116 and 117.

Accordingly, referring to FIGS. 3 and 4, the first and second upper restraining link parts 113 and 114 and the first and second lower restraining link parts 118 and 119 of FIG. 3 may perform a pitch rotation θ₁ and a yaw rotation θ₂ as illustrated in FIG. 4.

As illustrated in FIG. 3, according to the present disclosure, by having the double delta structure, a structure resistant to torsion (a moment) or external force is realized.

That is, a first delta structure is formed by the link coupling of the first and second upper joint parts and the first and second upper restraining link parts disposed on the upper sides of the first, second, and third link parts 100, 200, and 300, respectively, and a second delta structure is formed by the link coupling of the first and second lower joint parts and the first and second lower restraining link parts disposed on the lower sides of the first, second, and third link parts 100, 200, and 300, respectively. Accordingly, the first delta structure and the second delta structure are configured parallel to each other vertically.

In addition, a parallelogram is formed by the first and second upper restraining link parts and the first and second lower restraining link parts disposed on the upper sides and lower sides of the first, second, and third link parts 100, 200, and 300. That is, as illustrated in FIG. 3, a first parallelogram 41 and a second parallelogram 42 are formed by the first and second upper restraining link parts 113 and 114 and the first and second lower restraining link parts 118 and 119 of the first link part 100. Accordingly, first and second parallelograms are formed on each of the second link part 200 and the third link part 300.

Meanwhile, as illustrated in FIG. 3, a moving shaft coupling part 130 of the first link part 100 is provided on one side of the second base link part 120, and is directly or indirectly coupled to a moving shaft 10 so that the second base link part 120 is movable along the moving shaft 10.

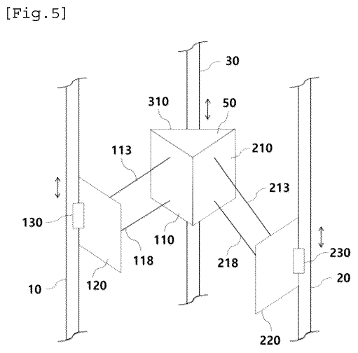

The double delta structure of a surgical robot according to an embodiment of the present disclosure may, for example, move as illustrated in FIG. 5.

That is, the moving shaft coupling part 130 of the second base link part 120 of the first link part 100 is directly or indirectly coupled to a first moving shaft 10, so the second base link part 120 moves up and down along the first moving shaft 10.

A moving shaft coupling part 230 of a second base link part 220 of the second link part 200 is directly or indirectly coupled to a second moving shaft 20, so the second base link part 220 moves up and down along the second moving shaft 20.

A moving shaft coupling part 330 of a second base link part 320 of the third link part 300 is directly or indirectly coupled to a third moving shaft 30, so the second base link part 320 moves up and down along the third moving shaft 30.

By moving each of the second base link part 120 of the first link part 100, the second base link part 220 of the second link part 200, and the second base link part 320 of the third link part 300 up and down by the same or different distance, the moving frame 50 may be moved with three degrees of freedom.

Meanwhile, the length of each link of the first and second upper restraining link parts and the first and second lower restraining link parts of the first link part 100, the first and second upper restraining link parts and the first and second lower restraining link parts of the second link part 200, and the first and second upper restraining link parts and the first and second lower restraining link parts of the third link part 300 may be different from each other.

In describing the present disclosure, description of prior arts and matters that are obvious to those skilled in the art may be omitted, and the omitted descriptions of components (methods) and functions may be sufficiently referenced within the scope of the technical idea of the present disclosure. In addition, the components of the present disclosure described above are explained for the convenience of explanation of the present disclosure, and components not described here may be added as long as they do not deviate from the technical idea of the present disclosure.

The description of the component and function of each part described above is explained separately from each other for convenience of explanation, and if necessary, one component and function thereof may be implemented by integrating with another component, or may be implemented in a more subdivided manner.

Above, the description has been made with reference to an embodiment, but the present disclosure is not limited thereto, and various modifications and applications are possible. That is, those skilled in the art will be able to easily understand that many modifications are possible without departing from the gist of the present disclosure. In addition, it should be noted that when it is determined that detailed description of known functions and components related to the present disclosure, or detailed description of the relationship of the combination of each component of the present disclosure may unnecessarily obscure the gist of the present disclosure, the detailed description thereof is omitted.

### DESCRIPTION OF THE REFERENCE NUMERALS IN THE DRAWINGS

10: First moving shaft
20: Second moving shaft
30: Third moving shaft
41: First parallelogram
42: Second parallelogram
50: Moving frame
100: First link part
110: First base link part
111: First upper joint part
112: Second upper joint part
113: First upper restraining link part
114: Second upper restraining link part
116: First lower joint part
117: Second lower joint part
118: First lower restraining link part
119: Second lower restraining link part
120: Second base link part
130: Moving shaft coupling part
200: Second link part
210: First base link part
211: First upper joint part
212: Second upper joint part
213: First upper restraining link part
214: Second upper restraining link part
216: First lower joint part
217: Second lower joint part
218: First lower restraining link part
219: Second lower restraining link part
220: Second base link part
230: Moving shaft coupling part
300: Third link part
310: First base link part
311: First upper joint part
312: Second upper joint part
313: First upper restraining link part
314: Second upper restraining link part
317: Second lower joint part
318: First lower restraining link part
319: Second lower restraining link part
320: Second base link part
330: Moving shaft coupling part

1. A double delta structure of a surgical robot, the structure comprising:
a first link part link-coupled to perform pitch and yaw rotations around a reference axis in a first direction;
a second link part link-coupled to perform pitch and yaw rotations around a reference axis in a second direction; and
a third link part link-coupled to perform pitch and yaw rotations around a reference axis in a third direction.

2. The structure of claim 1, wherein a first delta structure is formed through link connections of upper sides of the first, second, and third link parts, and a second delta structure is formed through link connections of lower sides of the first, second, and third link parts so as to form a double delta structure.

3. The structure of claim 2, wherein each of the first, second, and third link parts is composed of identical pairs of links and is link-coupled to form a parallelogram shape.

4. The structure of claim 3, wherein each of the first, second, and third link parts is link-coupled such that a plurality of parallelogram shapes are parallel to each other.

5. The structure of claim 3, wherein first, second, and third parallelograms formed on each of the first, second, and third link parts are formed to face each other while being spaced apart from each other at predetermined angles.

6. The structure of claim 1, wherein each of the first, second, and third link parts comprises:
a plurality of base link parts;
a plurality of upper joint parts link-coupled to rotate on upper sides of the base link parts;
a plurality of upper restraining link parts link-coupled to the upper joint parts to perform pitch and yaw rotations;
a plurality of lower joint parts link-coupled to rotate on lower sides of the base link parts; and
a plurality of lower restraining link parts link-coupled to the lower joint parts to perform pitch and yaw rotations.

7. The structure of claim 6, wherein first and second upper restraining link parts are link-coupled parallel to each other to the plurality of upper joint parts to perform pitch and yaw rotations; and
first and second lower restraining link parts are link-coupled parallel to each other to the plurality of lower joint parts to perform pitch and yaw rotations.

8. The structure of claim 7, further comprising:
a first parallelogram formed by comprising the first upper restraining link part and the first lower restraining link part; and
a second parallelogram formed by comprising the second upper restraining link part and the second lower restraining link part.

9. The structure of claim 8, wherein the first parallelogram and the second parallelogram are formed parallel to each other.

10. The structure of claim 6, wherein the plurality of base link parts comprises:
a first base link part to which a first upper joint part and a first lower joint part are link-coupled to be rotatable; and
a second base link part to which a second upper joint part and a second lower joint part are link-coupled to be rotatable.

11. The structure of claim 10, wherein opposite-side coupling links of first and second upper restraining link parts are link-coupled to the first and second upper joint parts, respectively, and opposite-side coupling links of first and second lower restraining link parts are link-coupled to the first and second lower joint parts, respectively.

12. The structure of claim 11, wherein the second base link part further comprises a moving shaft coupling part that moves along a moving shaft, and
a moving frame formed by combination of the first, second, and third link parts is capable of performing three degrees of freedom movement by moving along the moving shaft.
